(19) United States
(12) Patent Application Publication
(10) Pub. No.: US 2007/0043142 A1
(43) Pub. Date: Feb. 22, 2007

(54) DENTAL COMPOSITIONS BASED ON NANOFIBER REINFORCEMENT

(76) Inventors: Hanna Dodiuk-Kenig, Haifa (IL); Barry Zalsman, Yuad (IL); Kira Lisenboim, Raanana (IL); William A. McHale, Collegeville, PA (US)

Correspondence Address: rudoler & derosa llc;ATTN: DOCKET CLERK, 2 BALA PLAZA,, SUITE 300, BALA CYNWYD, PA, 19004, US

(21) Appl. No.: 11/465,690

(22) Filed: Aug. 18, 2006

Related U.S. Application Data
(60) Provisional application No. 60/709,843, filed on Aug. 19, 2005.
(60) Provisional application No. 60/813,219, filed on Jun. 13, 2006.

(51) Int. Cl. A61K 6/08

(52) U.S. Cl. 523/116

(57) ABSTRACT

A dental material using nano material that will serve as reinforcement and will enhance mechanical properties with minimal sacrifice in other properties including processability of a dental material. The dental material may be used as a filling, restorative, cement, liner, adhesive or primer. This is achieved by combining several polymerizable monomers and/or oligomers, a polymerization initiator, at least one hyperbranched additive and at least one of an electrospun nanofiber, an electrospun nanosphere or a hyperbranched macromolecule. The hyperbranched additive may be hyperbranched molecules, dendridic molecules (such as dendrimers). In a preferred embodiment a caged silica (such as POSS) is used for a caged macromolecule. The material may also include nanoclay or traditional composite fillers. The material may optionally include accelerators (such as DEHPT), cross linkers or pigment

### CROSS REFERENCE TO RELATED APPLICATIONS

The present application claims the benefit of U.S. Provisional Application No. 60,709,843 filed on Aug. 19, 2005 and U.S. Provisional Application No. 60/813,219 filed on Jun. 13, 2006, which are incorporated herein by reference.

### STATEMENT REGARDING FEDERALLY SPONSORED RESEARCH OR DEVELOPMENT

The present invention was not developed with the use of any Federal Funds, but was developed independently by the inventors.

### BACKGROUND OF THE INVENTION

Polymerizable compositions have various use in dentistry, for example as materials for reconstructing teeth or as adhesive for holding reconstructive elements in place. Such compositions generally include a hydrophobic resin and an inert filler, such as quartz or silica-glass. Often the filler particles are coated with a coupling agent to bond to the resin matrix. The strength of composites is dependent on chemical and Van der Waals interfacial forces between the polymer matrix and filler particles. These forces may be enhanced by the presence of polar functional groups on the polymer and/or by the treatment of filler surfaces with silanes, titanates, or other surface-active agents (Carrera, Polymer Chemistry. An Introduction, Fourth ed. Marcel Dekker, Inc 1999). Particle size and shape, as well as derived properties like specific surface and particle packing, are the most significant factors affecting the mechanical characteristics of a compound. The polymerizable compositions are usually cured by free radical polymerization, which may be initiated using visible light irradiation (often referred to as “visible light curing” or simply “light curing”) or by an oxidation-reduction reaction (sometimes called “self-curing”). Although compositions are known having acceptable compressive or flexural strength, such compositions also have one or more undesirable qualities.

For example, International Patent Application WO 98/36729 to Klee et al., discloses polymerizable compositions for forming dental materials, comprising a polymerizable resin consisting of a methacryloyl terminated hyperbranched polymer, a polymerizable monomer which was specially synthesized by inventors, a filler, and at least one polymerization initiator, sensibilizer or stabilizer. These compositions are reported to yield dental materials with a shrinkage of less than 1.5% when polymerized under pressure but with a shrinkage at the range of 1.98% to 2.89% when polymerized without pressure. The material stiffens upon application of shear stress or pressure and does not relax within a predetermined working time, due to its rheopex rheologic behavior. Furthermore, the compressive strength of the materials obtained is less than 250 MPa.

Another example of the difficulty in developing polymerizable dental compositions having desired qualities is illustrated by U.S. Pat. No. 5,886,064 to Rheinberg et al. It is known in the art to increase the amount of inert filler so as to increase the strength of the cured composition, but often increasing the fill content leads to loss of moldability of the composition, which makes placing it and forming it into the proper shape in the mouth of the patient difficult. To address this difficulty, U.S. Pat. No. 5,886,064 discloses a polymerizable composition which becomes flowable under compressive or shear stress. The inventors state that the composition can be packed in similar manner to amalgam and is particularly suitable as dental material or for the production of a dental material. This is achieved by combining a polymerizable monomer and/or oligomer, a polymerization initiator, a filler, and a dendrimer, where the dendrimer is a propylenimine, a polyether, a polythioether, a polyphenylenamide, or a polyphenylene ester dendrimer. The composition contains at least 70 wt. % of filler and 0.5 to 28 wt. % of dendrimer, and becomes flowable under pressure and/or shear stress. However, the composition demonstrates compressing strength of around only 170 MPa and rather high values of shrinkage.

Dendritic molecules, such as those used in U.S. Pat. No. 5,886,064, are known in the art. For example, U.S. Pat. No. 5,610,268 to Meijer et al., relates to dendrimers whose branches are formed by vinyl cyanide units, and to processes for their production. These dendrimers are suitable inter alia for mixing with thermoplastic polymers or polymeric compositions. Dendrimers with polymerizable groups or highly-filled mixtures are not mentioned. Likewise, U.S. Pat. No. 5,418,301 to Hult et al. relates to dendritic macromolecules based or polyesters, which are characterized by a highly-branched (hyper-branched) structure rather than ideally branched dendrimer structure, and to processes for their production. The dendritic macromolecules are disclosed in U.S. Pat. No. 5,418,301 as being suitable inter alia as a component for polymerizable compositions, although only liquid varnishes are described while filler-containing compositions are not disclosed.

### BRIEF DESCRIPTION OF THE INVENTION

A dental material using nano material that will serve as reinforcement and will enhance mechanical properties with minimal sacrifice in other properties including processability of a dental material. This is achieved by combining several polymerizable monomers and/or oligomers, a polymerization initiator, at least one hyperbranched additive and at least one of an electrospun nanofiber, an electrospun nanosphere or a hyperbranched macromolecule. The hyperbranched additive may be hyperbranched molecules or dendridic molecules (such as dendrimers). In a preferred embodiment a caged silica (such as POSS) is used for a caged macromolecule. The material may also include nanoclay or traditional composite fillers. The material may optionally include accelerators (such as DEHPT), cross linkers or pigment (for colors). The electrospun nanofiber or electrospun nanosphere may be processed from silk, cellulose, starch, polyamides, carbon, silica, alumina, zirconia, polyurethanes, polyesters, polylactides (PLLA), polyolefins, collagen, polyvinyl alchohol (PVOH), polylacticacid, polyglycolic. The dental material may be used as a filling, restorative, cement liner, adhesive or primer.

### DETAILED DESCRIPTION OF THE INVENTION

There is thus provided, in accordance with a preferred embodiment of the present invention, a polymerizable composition which comprises a plurality of polymerizable monomers, a polymerization initiator, at least one filler, and a polymerizable resin comprising a thermoplastic resin and a dendritic molecule, and optionally a cross-linked, wherein said composition contains at least about 40-95 wt. % of the filler, and from about 0.1 to about 10.0 wt. % of the dendritic molecule and 0.01% wt. nano-fibers.

In a preferred embodiment of the invention, the polymerizable monomer is chosen from the group consisting of mono- and multifunctional acrylates or methacrylates, preferably methyl methacrylate, triethylene glycol dimethacrylate (TEDMA), 2-hydroxyethyl methacrylate, hexanediol methacrylate, or dodecanediol dimethacrylate.

In one preferred embodiment of the invention, the monomer is substantially the only monomer present. In another preferred embodiment of the invention, the monomer is present as part of a mixture of monomers. The monomer is polymerizable by free radical polymerization. In one preferred embodiment of the invention, the free radical polymerization may be initiated by visible light radiation. In another preferred embodiment of the invention, the free radical polymerization may be initiated by an oxidation-reduction reaction, preferably by reaction of an amine with a peroxide. In a preferred embodiment of the invention, the monomer contains one or more functional groups selected from the group consisting of urethane, amine, acrylic, carboxylic, amide and hydroxyl. In a preferred embodiment of the invention, the at least one monomer is present in the composition in an amount of between about 12 and about 20 wt. %.

In a preferred embodiment of the invention. The thermoplastic resin is comprised of the group consisting of bisphenol-A-dimethacrylate, bisphenylglycidyl methacrylate (Bis-GMA), mono- and multi-functional aliphatic and aromatic urethane acrylate oligomers, epoxy-acrylate oligomers, urethane di-methacrylate or urethano-acrylate oligomers. It should be noted that the thermoplastic resin is actually the result of the polymerization of the monomers and/or oligomers that it is comprised of, although in some embodiments such resin may also be added to begin with. Preferably, units of which the thermoplastic resin is composed have an average moleular weight (MW) of between about 500 and about 3000. In one preferred embodiment of the invention, the thermoplastic resin comprises substantially only one type of oligomer. In another preferred embodiment of the invention, the thermoplastic resin comprises a mixture of oligomers. In one preferred embodiment of the invention, the free radical polymerization may be initiated by visible light radiation. In another preferred embodiment of the invention, the free radical polymerization may be initiated by an oxidation-reduction reaction, preferably by reaction of an amine with a peroxide. In a preferred embodiment of the invention, the thermoplastic resin contains one or more functional groups selected from the group consisting of urethane, amine, acrylic, amide, and hydroxyl. In a preferred embodiment of the invention, the thermoplastic resin is present in the composition in an amount of between about 10 and about 18 wt. %.

In a preferred embodiment of the invention, the dendritic molecule is a dendrimer. In a preferred embodiment, the dendrimer has from about 1 to about 20 generations of at least one monomeric or polymeric branching chain extender. In a preferred embodiment of the invention, the terminal units of the dendrimer contain functional groups which can react with functional groups on the monomer, the thermoplastic resin or the cross-linker. In a preferred embodiment of the invention, the dendrimer has a molecular weight between about 1,500 and about 25,000.

In another preferred embodiment of the invention, the dendritic molecule is a hyperbranched molecule. In a preferred embodiment, the hyperbranched molecule has from about 1 to about 20 generations. In a preferred embodiment, the hyperbranched molecule has at least one terminal unit which can react with a functional group on at least one of the monomer, the thermoplastic resin or the crosslinker. In a preferred embodiment of the invention, the hyperbranched molecule contains functional groups selected from the group consisting of hydroxyl, amine, carboxylic, ester, amide, sulfide, carboxylate, fatty acid and any reactive functional group. In a preferred embodiment of the invention, the hyperbranched molecule has a molecular weight between about 1,500 and about 25,000.

In a preferred embodiment of the invention, the filler nanofiber or nanosphere is selected from the group consisting of carbon, silica, alumina and other glass oxides and ceramics, or thermoplastic polymers like nylon, polyurethanes, polyvinyl alcohol (PVOH), polylacticacid, polyglycolic-acid and copolymer of those, silk, cellulose and the like, natural as well as synthetic polymeric nanofiber. The nanofiber may be treated by special surface treatment based on sylanization reaction, preferably having an average diameter of between about 1 nm and 300 nm. In one preferred embodiment of the invention the nanofiber filler are coated with a coupling agent to bond to the resin matrix, preferably with a coating containing silyl groups or the nanofiber filler are uncoated. In another preferred embodiment, prior to mixing in the composition of the invention the nanofiber filler are optionally treated with hyperbranched polymers or dendrimers in order to enhance interfacial adhesion to the resin matrix.

In a preferred embodiment of the invention, the composition comprises an oxidizing initiator selected from the group consisting of benzoyl peroxide, lauryl peroxide, benzoin, benzophenone, alpha-diketones. In a preferred embodiment, the oxidizing initiator is present in an amount of between about 0.3 and 1.5 wt. %. A preferred oxidizing initiator for use in self-cured polymerization is benzoyl peroxide. A preferred oxidizing initiator for use in photopolymerization is camphor quinone.

In a preferred embodiment of the invention, the composition also comprises a reducing initiator selected from the group consisting of tertiary amines. Reducing initiators are preferably used as reducing agents in combination with oxidizing initiators such as benzoyl peroxide, lauryl peroxide, or α-diketones, to effect more rapid generation of radicals. Preferred reducing initiators for self-cured polymerization are N,N-dimethyl-p-toluidine and N,N-dimethyl-sym-xylidine. Preferred reducing initiators for use in photopolymerization are ethyl-4-dimethyl-aminobenzoate (EDB) and diethyl-aminoethyl methacrylate. Preferably, the ratio of photoiniator to amine is about 1:1.

In a preferred embodiment of the invention, the composition comprises a cross-linker. The inclusion of a cross-linker is especially preferably when the composition will be polymerized to function as an adhesive. In a preferred embodiment, the cross-linker contains functional groups which can cross-link one or more of the monomer, oligomer and dendritic molecule. In a preferred embodiment, the cross-linker contains functional groups selected from the group consisting of hydroxyl and acrylic. In a preferred embodiment, the cross linker is selected from the group consisting of multifunctional acrylates, preferably tri- or tetrafunctional acrylates. In a preferred embodiment, the cross linker is present in the composition in an amount of between about 0.5 and 2.0 wt. %.

In a preferred embodiment of the invention, a filler is selected from the group consisting of quartz or silica-glass. Silica-glass preferably containes strontium, barium, zinc, boron and yttrium, aluminoborosilicate glass, colloidal silica or various other types of silica. In a preferred embodiment the caged macromolecule is polyhedral oligomeric silsequioxanes (POSS). POSS are nonostructed organic/inorganic hybrid compounds that have been used as reactive nanofillers to form nanocomposites. Silsesquioxanes are a class of compounds with the empirical formula RSiO1.5. The caged silica may possess a variety of functional groups (R group) that can potentially react with the host matrix.

A variety of POSS structures from cage size 6 through 12 are available, generally, the cage size 8 is mostly used. POSS monomers are designed to be copolymerizes or grafted into/onto the polymer chains to provide molecular level reinforcement. There is no limit to the type of functionality that can be placed on the cage, anywhere from one to eight groups.

Several commercial hyperbranched additives are available on the market such as HYBRANE (made by DSM of The Netherlands) and BOLTORN (made by Perstorp Corp. of Sweden). Other suitable hyperbranched additives are hyperbranched polyesteramide (such as those described in U.S. Pat. Nos. 6,387,496 and 6,392,006) and hyperbranched polyester. The hyperbranched additive may be a hyperbranched or dendritic macromolecule built up of hydroxyl units. The hydroxyl units may be combined with amide molecules having nitrogen atoms as branching points. Likewise the hyperbranched additive may be a hyperbranched or dendritic macromolecule with a reactive group, wherein the reactive group is comprised of hydroxyl, amine, carboylic, ester, amide, sulfide, carboxylate or fatty acid.

Suitable electrospun nanofibers include fibers spun from polyvinyl alcohol (PVOH), poly-l-lactic acid (PLLA) and polyamides (PA). Suitable electrospun nanospheres include spheres spun from PVOH.

Commonly used monomer suitable for the invention are bisphenylglycidyl methacrylate (Bis-GMA), triethylene glycol dimethacrylate (TEGDMA), 2-hydroxyethyl methacrylate, hexanediol methacrylate, or dodecanediol dimethacrylate, bisphenol-A-dimethacrylate, 2,6-di-tert-butyl-4-methylphenol (BHT), 2-hydroxyethylmethacrylate (HEMA) or N,N-dimethyl-p-toluidine. Molecules built from Bis-GMA such as those described in U.S. Patent Application Publications 2006/0058415 and 2006/0058418 are also suitable for the invention.

Preferably, the filler is in the form of particles, preferably having an average diameter of between about 30 nm and 30 μm. In one preferred embodiment of the invention the filler particles are coated with a coupling agent to bond to the resin matrix, preferably with a coating containing silyl groups (sometimes referred to as “silanized” filler as is known in the art). In another preferred embodiment of the invention the filler particles are uncoated. In another preferred embodiment, prior to mixing in the composition of the invention the filler particles are optionally treated with hyperbranched polymers or dendrimers in order to enhance interfacial adhesion to the resin matrix and nono-fibers for reinforcement of the nano-composite. In a preferred embodiment of the invention, the filler contains matter which is radiopaque.

There is also provided, in accordance with a preferred embodiment of the present invention, a process for forming a dental material, comprising the steps of (a) providing a polymerizable composition comprising at least one polymerizable monomer, a polymerization initiator, at least one filler, and a polymerizable resin comprising a thermoplastic resin and optionally a cross-linker wherein said composition contains at least about 40-95 wt. % of the filler, and from about 0.1 to about 10.0 wt. % of the dendritic molecule and (b) polymerizing said composition.

In one preferred embodiment of the invention, the material formed is a dental composite. In another preferred embodiment of the invention, the material formed is a dental adhesive.

There is also provided, in accordance with a preferred embodiment of the present invention, a denial material having a compressive strength of at least about 200 MPa, preferably at least about 250 MPa as determined by the method of ISO 9917 and linear shrinkage of less than about 2%, preferably less than about 1.5%, the dental material being the result of polymerization of a composition comprising at least one polymerizable monomer, a polymerization initiator, at least one filler, and a polymerizable resin comprising a thermoplastic resin and a dendritic molecule, and optionally a cross-linker, wherein said composition contains at least about 40-95 wt. % of the filler, and from about 0.1 to about 10.0 wt. % of the dendritic molecule.

There is also provided, in accordance with a preferred embodiment of the invention, a primer for pre-treating a tooth or other dental surface prior to application of an adhesive to said dental surface, comprising a solvent acceptable for use in dentistry and between about 1-30 wt. % of a hyperbranched dendritic macromolecule having a core which is built up by polycondensation so that the hyperbranched molecule has functional groups, for example, hydroxyl units in the terminal units and has amide nitrogen atoms as branching points.

There is also provided, in accordance with a preferred embodiment of the invention, a process for pre-treating a tooth or other dental surface prior to application of an adhesive to said tooth or dental surface, comprising (a) providing a solution comprising a solvent acceptable for use in dentistry and between about 1-30 wt. % of a hyperbranched dendritic macromolecule having a core which is built up by polycondensation of cyclic anhydrides with diisopropanolamine, so that the hyperbranched molecule has hydroxyl units in the terminal units and has amide nitrogen atoms as branching points, and (b) applying said solution to said tooth or other dental surface. In addition, 0.01-5% wt. nano-fibers of various type (see above) can be added.

A nanomaterial, such as a nanoclay may also be used in the dental material. One such type of nanoclay is alkyl quaternary ammonium bentonite also known by its trade name of CLOISITE and manufactured by Southern Clay Products.

It preferred embodiments, the present invention provides polymerizable compositions which yield dental materials having improved compressive strength and shrinkage properties vis-a-vis dental materials known in the prior art. In additional preferred embodiments of the invention, the dental materials may be formulated to have additional improved properties, such as water sorption or bonding to tooth substrates as expressed in measured shear bond strength.

A common feature to all the preferred embodiments of the present invention is the incorporation into the polymerizable composition of an amount of a dendritic polymer combined with nano-fibers which upon curing is effective to impart to the composition, in conjunction with the other components in the composition, the desired properties of compressive strength and shrinkage.

Thus, for example, suitable combinations of monomers, thermoplastic resins and mono- or/and multifunctional acrylates or methacrylates such as methyl methacrylate, triethylene glycol dimethacrylate, 2-hydroxyethyl methacrylate, hexanediol methacrylate, dodecanediol dimethacrylate, as the monomer, bisphenol-A-dimethacrylate, bisphenylglycidyl methacrylate, mono- and multi-functional aliphatic and aromatic urethane acrylate oligomers, epoxy-acrylate oligomers and urethano-acrylate oligomers, preferably having MW between 500 and 3000 as the thermoplastic resin, and dendritic molecules having functional groups selected from the group consisting of hydroxyl, amine, carboylic, ester amide, sulfide, carboxylate and fatty acid as the terminal groups. It has been found that when the dendritic molecule used is a dendrimer, it is preferably for the dendrimer to have between about 3 and about 770 terminal groups, and/or to contain between about 0 and 8 generations. Preferably, when the dendritic molecule used is a hyperbranched polymer, the hyperbranched polymer has a degree of branching between about 0.4 and 0.9. In preferred embodiments of the invention, the interior the dendritic molecule is built up from units containing hydroxyl or amine groups.

Examples of pairs of initiators suitable for use in accordance with the present invention are benzoyl peroxide, camphor quinone as oxidizing initiators and amines, preferably N,N-dimethyl-p-toluidine, ethyl-4-dimethylaminobenzoate and their derivatives, as reducing initiators. When cross-liners are used, these are preferably molecules capable of cross-linking the groups B on the terminii of the dendritic molecules with the thermoplastic resin and/or the at least one monomer. Preferably, the initiator and cross-linker are each independently present in an amount of between about 0.3 and about 1.5 wt. %.

Examples of fillers suitable for use in accordance with the present invention are silanized glass and other dental fillers as are well known in the art, such as, quartz or silica-glass containing at least one of strontium, barium, zinc, boron, and yttrium, aluminoborosilicate glass, and colloidal silica and caged silica (POSS). Preferably, the fillers are coated with a dendritic molecule, preferably the same dendritic molecule used in the remainder of the composition of the invention. The fillers preferably have an average particle size of between about 10 nm and about 30 μm, and may be present a mixture of particles having a range of sizes.

It has been found that dental materials prepared in accordance with the present invention exhibit low shrinkage, generally below about 2.0% and preferably below about 1.5%, measured by the method described below. At the same time, and in contrast to dental materials known in the prior art, including those prior art dental materials prepared from mixtures of monomers and/or oligomers and dendritic polymers, the dental materials obtained in accordance with the present invention also exhibit good compressive strength, generally at least about 200 MPa and preferably at least about 300 MPa.

In one preferred embodiment of the present invention, a tooth or other dental surface to which an adhesive is to be applied may be pre-treated with a dendritic polymer as described above. Such application may be made, for example, by contacting the tooth or dental surface with a solution containing from about 1 to about 30% dendritic polymer and 0.01-5% wt. nano-fibers in a dentally acceptable solvent, such as ethanol or another alcohol or propylene glycol or another glycol.

Examples of some preferred embodiments of the invention will now be illustrated through the following illustrative and non-limitative example.

### EXAMPLE 1

### Composition Without Dendritic Molecule for Use as Core Build-Up Material

A highly filled dental cement is formed from a composition consisting of two parts, mixture A (Base) and mixture B (Catalyst) which are mixed in equal amounts and oxidatively polymerized.

Mixture A: To a mixture of 1.4000 g of bishpenylglycidylmethacrylate (Bis-GMA), 1.7 mg 2,6-di-tert-butyl-4-methylphenol (BHT) and 1.5000 g 2-hydroxyethylmethacrylate (HEMA) were added 0.0400 g of N,N-dimethyl-p-toluidine and 7.0583 g of silanised glass filler at room temperature. This mixture was then ground.

Mixture B: To a mixture of 1.3400 g of bisphenylglycidylmethacrylate (Bis-GMA), 2.0 mg BHT and 1.3080 g tetraethylglycidylmethacrylate (TEGDMA) were added 0.0400 g of benzoyl peroxide and 7.3100 g of silanised glass filler at room temperature. This mixture was then ground.

Mixtures A and B were stored separately for at least 24 hours at room temperature prior to use.

A dental cement was prepared by polymerizing a mixture consisting of 2.500 g of Mixture A and 2.500 g of Mixture A.

### EXAMPLE 2

The procedure of Example 1 was followed except that in each of mixtures A and B, 0.01 g of Bis-GMA (representing 0.1 wt. % of the total weight of each mixture) was replaced with a dendripolyamide oligomer based on a six-valent semi-flexible core (Molecular Weight 12,100; H-functionality size 45 mole⁻¹; H-functionality type as Versamide 125). The compressive strength of the resulting cement was found to be in the range of 150.0±20.0 MPa. Water sorption was at the range of 16.0±2.0 μg/mm³. The result complies with ISO 4049:2000(E) requirements. Linear shrinkage was in the range of ±3.6%.

### EXAMPLE 3

The procedure of Example 1 was followed, except that in each of mixtures A and B, 0.01 g of Bis-GMA (representing 0.1 wt. % of the total weight of each mixture) was replaced with a hyperbranched polyesteramide. The compressive strength of the resulting cement was found to be in the range of 303.7±20.0 MPa. Water sorption was found to be within ISO 4049:2000(E) requirements. Linear shrinkage determined as described in Example 1 was ±0.8%.

### EXAMPLE 4

The procedure of Example 3 was followed, except that 0.03 g of the same hyperbranched polyesteramide used in Example 3 (representing 0.3 wt. % of the total weight of each mixture) was used in each of Mixtures A and B. The compressive strength of the resulting cement was found to be in the range of 386.0±20.0 MPa. Water sorption was found to be within ISO 4049:2000(E) requirements. Linear shrinkage determined as describe in Example 1 was ±1.5%.

### EXAMPLE 5

The procedure of Example 3 was followed, except that 0.05 g of the hyperbranched polyesteramide (representing 0.5 wt. % of the total weight of each mixture) was used in each of Mixtures A and B. The compressive strength of the resulting cement was found to be in the range of 227.0±20.0 MPa. Water sorption was at the range of 16.0±2.0 μg/mm³. Linear shrinkage was in the range ±2.3%.

### EXAMPLE 6

The procedure of Example 1 was followed, except that in each of mixtures A and B, 0.05 g of Bis-GMA (representing 0.5 wt. % of the total weight of each mixture) was replaced with a dendripolyamide oligomer with a four-valent semi-flexible core (Molecular Weight 6,500; H-functionality size 30 mole⁻¹; H-functionality type as Versamide 125). The compressive strength of the resulting cement was found to be in the range of 201.0±20.0 MPa. Water sorption determined was at the range of 30.0±2.0 μg/mm³. Linear shrinkage determined as described in Example 1 was at the range ±2.4%.

### EXAMPLE 7

### Q-Core Composite (BJM)—Commercial Core Build-Up Material Chemically Cured

Two mixtures, Mixture A and Mixture B, were prepared as follows:

Mixture A: To a mixture of 1.3600 g of bisphenylglycidylmethacrylate (Bis-GMA), 1.7 mg 2,6-di-tert-butyl-4-methylphenol (BHT), 0.0300 g by perbranched polyesteramide 1.5700 g of 2-hydroxyethylmethacrylate (HEMA) were added 0.0400 g of N,N-dimethyl-p-toluidine and 6.9983 g of filler containing a mixture of colloidal silica. silanised glass, borosilicate glass and fluorine-releasing filler at room temperature. The Core Composite composition consists of the same components as the model one, but the filler level is different for two parts of composition and contains silanized glass, colloidal silica, borosilicate glass mixture, and fluorine-releasing filler. The changes in filler content were dictated by aesthetic demands and desired additional properties, easy handling, thermal conductivity, fluorine-release etc. This mixture of components was then ground to form Mixture A.

Mixture B: To a mixture of 1.3400 g of Bis-GMA, 1.3 mg BHT, 0.0270 g hyperbranched polyestramide 1.3000 g of tetraethylglycidylmethacrylate (TEGDMA) were added 0.0400 g of benzoyl peroxide and 7.2917 g of filler containing a mixture colloidal silica, silanised glass, borosilicate glass and fluorine-releasing filler at room temperature. The mixture of components was ground to form Mixture B.

Mixtures A and B were stored separately for at least 24 hours at room temperature prior to use, and then 2.5 g of Mixture A was mixed with 2.5 of Mixture B and allowed to cure for 10 minutes.

The dental material obtained after curing was found to have a compressive strength of 250.0±20.0 MPa, linear shrinkage of 1.50±0.50%, and water sorption 23.8 μ/mm³.

A comparison between the dental material obtained in Example 7 and core build-up materials prepared from commercially available compositions was carried out under identical conditions. The results of physical and mechanical evaluations, measured as described above, are summarized in Table 1:

TABLE 1
Comparison of main physico-mechanical properties of chemically cured composites

| | | | Requirements | Material/Supplier | | | | |
|---|---|---|---|---|---|---|---|---|
| | | | According to | TiCore/ | | | Encore/ | |
| | | | ISO 4049: | EDS | CompCore/ | CorePaste/ | Centric | |
| # | Property | Units | 2000(E) | Inc. | Premier | DenMat | Inc. | Example 7 |
| 1 | Compressive | MPa | 120.0 | 193.0 | 198.5 | 137.8 | 182.0 | 250.0 ± 20.0 |
| | strength | | | | | | | |
| 2 | Length | % | 3.5 | 2.7 ± 1.5 | 4.8 ± 1.5 | 6.6 ± 1.3 | 6.1 ± 1.8 | 1.5 ± 0.5 |
| | shrinkage | | | | | | | |
| 3 | Water | μg/mm³ | <40.0 | 5.5 | 6.8 | 2.2 | 3.4 | 23.8 |
| | sorption | | | | | | | |
| 4 | Working | min. | 1.5 | 2.0 | 2.5 | 2.5 | 2.8 | 2.5 |
| | Time | | | | | | | |
| 5 | Setting Time | min. | 5.0 | 4.5 | 4.8 | 5.0 | 4.75 | 5.0 |
| 6 | Texp | ° C. | <41.0 | — | 36.0 | — | — | 39.0 ± 2.0 |

### EXAMPLE 8

### Q-Core Composite (BJM)—Commercial Core Build-Up Material Dual Cured

The procedure of Example 7 was followed, except that for each of mixtures A and B photoinitiators were added.

Mixtures A and B were stored separately for at least 24 hours at room temperature prior to use, and then 2.5 g of Mixture A was mixed with 2.5 of Mixture B and allowed to cure for 10 minutes.

The dental material obtained after curing was found to have a compressive strength of 251.0±20.0 MPa, linear shrinkage of 1.20±0.15%, and water sorption 30.0 μg/mm³.

A comparison between the dental material obtained in Example 8 and core build-up materials prepared from commercially available compositions was carried out under identical conditions. The results of physical and mechanical evaluations, measured as described above, are summarized in Table 2:

TABLE 2
Comparison of main physico-mechanical properties* of dual cured composites

| | | | Requirements | Material/Supplier | | | | |
|---|---|---|---|---|---|---|---|---|
| | | | According to | Build-it | | LuxaCore, | Absolute | Q-Core dual |
| | | | ISO 4049: | FR, Jeneric | ParaCore, | Zenith- | Dentin, | polymerized, BFM |
| # | Property | Units | 2000(E) | Pentron | Coltene | DMG | Parkell | Lab, Ltd. |
| 1 | Compressive | MPa | 120.0 | 221.0 | 230.0 | 260.0 | 235.7 | 251.0 |
| | strength | | | | | | | |
| 2 | Linear | % | 3.5 | 2.0 | 2.7 | 2.7 | 4.0 | 1.2 |
| | shrinkage | | | | | | | |
| 3 | Water | μg/mm³ | <40.0 | 16.8 | 24.0 | 31.5 | | 30.0 |
| | sorption | | | | | | | |
| 4 | Setting Time | min. | 5.0 | 5.0 | 5.0 | 5.0 | 3.5 | 4.5 |
| 5 | Texp | ° C. | <41.0 | 31.9 | 36.3 | — | 39.7 | 39.0 |

*Standard Deviation is ±10%.

### EXAMPLE 9

### Liquid Dental Adhesive Without Dendritic Polymer

A liquid light-curable dental adhesive was prepared by mixing 2.100 g of tetrathylglycidylmthacrylate (TEGDMA), 2.700 g 2-hydroxyethylmethacrylate (HEMA), 4.200 g urethane di-methacrylate oligomer, 0.500 g phosphonate as a bonding agent, 0.446 g triacrylate monomer as a cross-linking agent, 0.025 g ethyl-4-dimethylaminobenzoate (EDB) as a polymerization accelerator, and 0.029 g camphor quinone as a polymerization initiator and exposing to light of 450-500 nm wavelength, as described below.

After bonding and curing the sample, specimens were placed in water at 37° C. for 24 hours. The shear bond strength (SBS) of the dental adhesive was found to be 6.3±2.0 MPa.

### EXAMPLE 10

### Liquid Dental Adhesive with Dendritic Polymers

The procedure of Example 9 was repeated, except that 0.020 g (0.2 wt. %) of a hyperbranched polyesteramide was added to the adhesive composition. The shear bond strength (SBS) was 10.5±2.0 MPa.

### EXAMPLE 11

The procedure of Example 9 was repeated except that 0.065 g (0.65 of wt. %). of the hyperbranched polyesteramide was added to the adhesive composition. The shear bond strength (SBS) was found to be 11.6±20 MPa.

### EXAMPLE 12

The procedure of Example 9 was repeated, except that 0.150 g of the hyperbranched polyesteramide was added to the adhesive composition. The shear bond strength (SBS) was found to be 10.7±20 MPa.

### EXAMPLE 13

The procedure of Example 9 was repeated, except that 0.020 g of dendripolyamide oligomer with a six-valent semi-flexible core (Molecular Weight 12,100; H-functionality size 45 mole⁻¹; H-functionality type as Versamide 125) were added to the adhesive composition. The shear bond strength (SBS) was found to be 5.5±2.0 MPa.

### EXAMPLE 14

### Filled Dental Adhesive Composition Without Dendritic Polymer

Dental adhesives may be used for final cementation of crowns and bridges, for inlays and onlays, for posts and cores, for ceramic crowns and Maryland bridges, or for bonding metal, plastic or ceramic orthodontic attachments to teeth. Adhesives may also be used for amalgam restoration, veneering of alloys, and for the implantation of prostheses. This and the following two examples compare dental adhesives prepared without and with dendritic molecules. The adhesives are “dual curable”, i.e. polymerization may be initiated by combining the two component mixtures A and B of the adhesive, but the rate polymerization can be increased by exposing the combined components to light.

Two mixtures, Mixture A and Mixture B, were prepared as follows:

Mixture A: To a mixture of 1.240 g 2-hydroxyethylmethacrylate (HEMA), 3.660 g urethane di-methacrylate oligomer and 0.200 g triacrylate monomer cross-linking agent were added 0.030 g N,N-dihydroxyethyl-p-toluidine (DHEPT), 0.030 g camphor quinone, 0.030 g ethyl-4-dimethylaminobenzoate (EDB), and 4.810 g strontium-alumino-fluoro-silicate glass at room temperature. These components were then mixed to form Mixture A.

Mixture B: To a mixture of 2.200 g bisphenylglycidylmethacrylate (Bis-GMA), 0.200 g of triacrylate monomer cross-linking agent and 1.700 g tetraethylglycidylmethacrylate (TEGDMA) were added 0.080 g benzoyl peroxide, 0.200 g aromatic acrylate monomer derivative coupling agent and 5.620 g strontium-alumino-fluoro-silicate glass at room temperature. These components were then mixed to form Mixture B.

Mixtures A and B were stored separately for 24 hours at room temperature, and then 2.5 g of Mixture A was mixed with 2.5 g of Mixture B and allowed to cure for 1 hour.

The dental material obtained after curing was found to have a shear bond strength of 3.4±1.3 MPa and a compressive strength of 222.0±20.0 MPa.

### EXAMPLE 15

### Filled Dental Adhesive Composition with Dendritic Polymer

The procedure of Example 14 was repeated, except that 0.100 g (1.0 wt. %) of a hyperbranched polyesteramide was added to each of Mixtures A and B. The shear bond strength (SBS) measured as in Example 12 was 6.5±1.3 MPa and compressive strength measured as in Example 1 was 117.0±20.0 MPa.

### EXAMPLE 16

### Filled Dental Adhesive Composition with Dendritic Polymer

The procedure of Example 14 was repeated, except that 0.150 g (1.5 wt. %) of the hyperbranched polyesteramide was added to each of Mixtures A and B. SBS was found to be 5.0±1.3 MPa and compressive strength found to be 96.0±20.0 MPa.

### EXAMPLE 17

### Unfilled Dental Adhesive Composition with Dendritic Polymer

Two mixtures, Mixture A and Mixture B, were prepared as follows:

Mixture A: To a mixture of 3.840 g 2 tetraethylglycidylmethacrylate (TEGDMA), 5.630 g bisphenylglycidylmethacrylate (Bis-GMA) were added 0.180 g N,N-dihydroxyethyl-p-toluidine (DHEPT), 0.150 g camphor quinone, 0.110 g ethyl-4-dimethylaminobenzoate (EDB), and 0.090 g hyperbranched polyesteramide without the conventional strontium-alumino-fluoro-silicate glass filler, as at example 14, at room temperature. These components were then mixed to form Mixture A.

Mixture B: To a mixture of 5.920 g bisphenylglycidylmethacrylate (Bis-GMA), 3.880 g tetraethylglycidylmethacrylate (TEGDMA) were added 0.110 g benzoyl peroxide, 0.090 g hyperbranched polyesteramide and without the conventional strontium-alumino-fluoro-silicate glass filler, as at example 14, at room temperature. These components were then mixed to form Mixture B.

Mixtures A and B were stored separately for 24 hours at room temperature, and then 2.5 g of Mixture A was mixed with 2.5 g of Mixture B and allowed to cure for 1 hour.

### EXAMPLE 18

### Unfilled Dental Adhesive Composition with Dendritic Polymer and PVOH Nanofibers and Nanospheres

Two mixtures, Mixture A and Mixture B, were prepared as follows:

Mixture A: To a mixture of 3.840 g 2 tetraethylglycidylmethacrylate (TEGDMA), 5.630 g bisphenylglycidylmethacrylate (Bis-GMA) were added 0.180 g N,N-dihydroxyethyl-p-toluidine (DHEPT), 0.150 g camphor quinone, 0.110 g ethyl-4-dimethylaminobenzoate (EDB), and 0.090 g hyperbranched polyesteramide without the conventional strontium-alumino-fluoro-silicate glass filler at room temperature. These components were then mixed to form Mixture A.

To mixture A 0.01% of electrospun nano-fibers based on poly vinyl alcohol (PVOH) (2 various diameters) were incorporated by shear mixing.

Mixture B: To a mixture of 5.920 g bisphenylglycidylmethacrylate (Bis-GMA), 3.880 g tetraethylglycidylmethacrylate (TEGDMA) were added 0.110 g benzoyl peroxide, 0.090 g hyperbranched polyesteramide and without the conventional strontium-alumino-fluoro-silicate glass filler at room temperature. These components were then mixed to form Mixture B.

To mixture B 0.01% of electrospun nano-fibers based on PVOH (2 various diameters) were incorporated by shear mixing.

Mixtures A and B were stored separately for 24 hours at room temperature, and then 2.5 g of Mixture A was mixed with 2.5 g of Mixture B and allowed to cure for 1 hour.

Results (Compressive strengths, flexural strengths and linear shrinkage) are represented in Table 3-5 respectively.

The variants are based on the same formulation as Example 18 and its properties are given also in Table 3-5.

TABLE 3
Compressive strength of unfilled resin as a function of
PVOH nano-particles type and concentration.

| Example 18 | CS, MPa | CS, MPa | CS, MPa |
|---|---|---|---|
| with PVOH | (250 nm | (130 nm | (200 nm |
| Nano-fibers | Diameter | Diameter | Diameter |
| Addition, | Nano- | Nano- | Nano- |
| % wt. | fibers) | fibers) | spheres) |
| 0.01 | 121.4 | 140.7 | 137.2 |
| 0.05 | 213.9 | 126.4 | 206.3 |
| 0.1 | 138.2 | 100.5 | 145.3 |
| 0.3 | 89.9 | 94.1 | 115.6 |

TABLE 4
Flexural strength of unfilled resin as a function of
PVOH nano-particles type and concentration.

| Example 18 | FS, MPa | FS, MPa | FS, MPa |
|---|---|---|---|
| with PVOH | (250 nm | (130 nm | (250 nm |
| Nano-fibers | Diameter | Diameter | Diameter |
| Addition, | Nano- | Nano- | Nano- |
| % wt. | fibers) | fibers) | spheres) |
| 0.01 | 260.3 | 214.7 | 354.4 |
| 0.05 | 171.8 | 226.0 | 357.3 |
| 0.1 | 225.6 | 145.5 | 317.6 |
| 0.3 | 244.9 | 230.5 | 189.6 |

TABLE 5
Linear shrinkage of unfilled resin as a function of
PVOH nano-particles type and concentration.

| Example 18 | LS, % | LS, % | LS, % |
|---|---|---|---|
| with PVOH | (250 nm | (130 nm | (200 nm |
| Nano-fibers | Diameter | Diameter | Diameter |
| Addition, | Nano- | Nano- | Nano- |
| % wt. | fibers) | fibers) | spheres) |
| 0.01 | 1.2 | 3.1 | 2.0 |
| 0.05 | 2.0 | 2.8 | 2.8 |
| 0.1 | 3 | 2.3 | 3.0 |
| 0.3 | 2.2 | 2.4 | 2.0 |

### EXAMPLE 19

### Unfilled Dental Adhesive Compositions with Dendritic Polymer and PLLA Nanofibers

Two mixtures, Mixture A and Mixture B, were prepared as follows:

Mixture A: to a mixture of 3.840 g 2 tetraethylglycidylmethacrylate (TEGDMA), 5,630 g bisphenylglycidylmethacrylate (Bis-GMA) were added 0.180 g N,N-dihydroxyethyl-p-toluidine (DHEPT), 0.150 g camphor quinone, 0.110 g ethyl-4-dimethylaminobenzoate (EDB), and 0.090 g hyperbranched polyesteramide without the conventional strontium-alumino-fluoro-silicate glass filler, as at example 14, at room temperature. These components were then mixed to form Mixture A.

To mixture A 0.01% of electrospun nano-fibers based on poly-1-lactic acid (PLLA) (2 various diameters) were incorporated by shear mixing.

Mixture B: To a mixture of 5.920 g bisphenylglycidylmethacrylate (Bis-GMA), 3.880 g tetraethylglycidylmethacrylate (TEGDMA) were added 0.110 g benzoyl peroxide, 0.090 g hyperbranched polyesteramide and without the conventional strontium-alumino-fluoro-silicate glass fiber at room temperature. These components were then mixed to form Mixture B.

To mixture B 0.01% of electrospun nano-fibers based on poly-1-lactic acid (PLLA) (2 various diameters) were incorporated by shear mixing.

Mixtures A and B were stored separately for 24 hours at room temperature, and then 2.5 g of Mixture A was mixed with 2.5 g of Mixture B and allowed to cure for 1 hour.

Results (Compressive strengths, flexural strengths and linear shrinkage) are represented in Table 6-8 respectively.

The variants are based on the same formulation as Example 19 and its properties are given also in Table 6-8.

TABLE 6
Compressive strength of unfilled resin as a function of
PLLA nano-fibers diameter and concentration.

| Example 19 | | |
|---|---|---|
| with PVOH Nano- | CS, MPa (250 nm | CS, MPa (130 nm |
| fibers Addition, % wt. | Diameter Nano-fibers) | Diameter Nano-fibers) |
| 0.01% | 136.2 | 144.2 |
| 0.05% | 165.0 | 182.1 |
| 0.10% | 143.9 | 133.3 |
| 0.30% | 94.0 | 125.0 |

TABLE 7
Flexural strength of unfilled resin as a function of
PLLA nano-fibers diameter and concentration.

| Example 19 | | |
|---|---|---|
| with PVOH Nano-fibers | FS, MPa (250 nm | FS, MPa (130 nm |
| Addition, % wt. | Diameter Nano-fibers) | Diameter Nano-fibers) |
| 0.01% | 290.2 | 363.7 |
| 0.05% | 293.4 | 267.9 |
| 0.10% | 317.6 | 307.5 |
| 0.30% | 342.9 | 280.5 |

TABLE 8
Linear Shrinkage of unfilled resin as a function of
PLLA nano-fibers diameter and concentration.

| Example 19 | | |
|---|---|---|
| with PVOH Nano-fibers | LS, % (250 nm | LS, M % (130 nm |
| Addition, % wt. | Diameter Nano-fibers) | Diameter Nano-fibers) |
| 0.01% | 2.8 | 2.6 |
| 0.05% | 2.4 | 2.2 |
| 0.10% | 2.5 | 2.3 |
| 0.30% | 2.7 | 1.8 |

### EXAMPLE 20

### Unfilled Dental Adhesive Composition with Dendritic Polymer and PAG Nanofibers

Two mixtures, Mixture A and Mixture B, were prepared as follows:

Mixture A: To a mixture of 3.840 g 2 tetraethylglycidylmethacrylate (TEGDMA), 5.630 g bisphenylglycidylmethacrylate (Bis-GMA) were added 0.180 g N,N-dihydroxyethyl-p-toluidine (DHEPT), 0.150 g camphor quinone, 0.110 g ethyl-4-dimethylaminobenzoate (EDB), and 0.090 g hyperbranched polyesteramide without the conventional strontium-alumino-fluoro-silicate glass filler at room temperature. These components were then mixed to form Mixture A.

To mixture A 0.01% of electrospun nano-fibers based on polyamide 6 (PA6) were incorporated by shear mixing.

Mixture B: To a mixture of 5.920 g bisphenylglycidylmethacrylate (Bis-GMA), 3.880 g tetraethylglycidylmethacrylate (TEGDMA) were added 0.110 g benzoyl peroxide, 0.090 g hyperbranched polyesteramide and without the conventional strontium-alumino-fluoro-silicate glass filler at room temperature. These components were then mixed to form Mixture B.

To mixture B 0.01% of electrospun nano-fibers based on polyamide 6 (PA6) were incorporated by shear mixing.

Mixtures A and B were stored separately for 24 hours at room temperature, and then 2.5 g of Mixture A was mixed with 2.5 g of Mixture B and allowed to cure for 1 hour.

Results (Compressive strengths, flexural strengths and linear shrinkage) are represented in Table 9.

The variants are based on the same formulation as Example 20 and its properties are given also in table 9.

TABLE 9
Mechanical Properties of unfilled (example 20) resin
as a function of PA6 nano-fibers concentration.

| Example 20 with | | | |
|---|---|---|---|
| PA6 Nano-fibers | | | |
| (diameter 250 nm) | | | |
| Addition, % wt. | CS, MPa | FS, MPa | LS, % |
| 0.05 | 77.8 | 236.6 | 2.1 |
| 0.1 | 63.1 | 249.5 | 3.2 |
| 0.3 | 67.6 | 234.3 | 2.7 |

### EXAMPLE 21

### Unfilled Dental Adhesive Composition with Dendritic Polymer and Nanopheres Silica

Two mixtures, Mixture A and Mixture B, were prepared as follows:

Mixture A: To a mixture of 3.840 g 2 tetraethylglycidylmethacrylate (TEGDMA), 5.630 g bisphenylglycidylmethacrylate (Bis-GMA) containing 50 wt % of surface-modified, synthetic, SiO2-nanospheres of very small size (diameter 20 nm) and narrow particle size distribution were added 0.180 g N,N-dihydroxyethyl-p-toluidine (DHEPT), 0.150 g camphor quinone, 0.110 g ethyl-4-dimethylaminobenzoate (EDB), and 0.090 g hyperbranched polyesteramide without the conventional strontium-alumino-fluoro-silicate glass filler at room temperature. These components were then mixed to form Mixture A.

Mixture B: To a mixture of 5.920 g bisphenylglycidylmethacrylate (Bis-GMA), 3.880 g tetraethylglycidylmethacrylate (TEGDMA) containing 50 wt % of surface-modified synthetic SiO2-nanopheres of very small size (diameter 20 nm) and narrow particle size distribution were added 0.110 g benzoyl peroxide, 0.090 g hyperbranched polyesteramide and without the conventional strontium-alumino-fluoro-silicate glass filler at room temperature. These components were then mixed to form Mixture B.

Mixtures A and B were stored separately for 24 hours at room temperature, and then 2.5 g of Mixture A was mixed with 2.5 g of Mixture B and allowed to cure for 1 hour.

### EXAMPLE 22

### Unfilled Dental Adhesive Composition with Dendritic Polymer, Nanospheres Silica and Nanofibers

Two mixtures, Mixture A and Mixture B, were prepared as follows:

Mixture A: To a mixture of 3.840 g 2 tetraethylglycidylmethacrylate (TEGDMA), 5.630 g bisphenylglycidylmethacrylate (Bis-GMA) containing 50 wt % of surface-modified, synthetic, SiO2-nanospheres of very small size (diameter 20 nm) and narrow particle size distribution were added 0.180 g N,N-dihydroxyethyl-p-toluidine (DHEPT), 0.150 g camphor quinone, 0.110 g ethyl-4-dimethylaminobenzoate (EDB), and 0.090 g hyperbranched polyesteramide without the conventional strontium-alumino-fluoro-silicate glass filler at room temperature. These components were then mixed to form Mixture A.

To mixture A 0.01% of electrospun nano-fibers based on PVOH (2 various diameters) were incorporated by shear mixing.

Mixture B: To a mixture of 5.920 g bisphenylglycidylmethacrylate (Bis-GMA), 3.880 g tetraethylglycidylmethacrylate (TEGDMA) containing 50 wt % of surface-modified, synthetic SiO2-nanospheres of very small size (diameter 20 nm) and narrow particle size distribution were added 0.110 g benzoylperoxide, 0.090 g hyperbranched polyesteramide and without the conventional strontium-alumino-fluoro-silicate glass filler at room temperature. These components were then mixed to form Mixture B.

To mixture B 0.01% of electrospun nano-fibers based on 250 nm diameter PVOH were incorporated by shear mixing.

Mixtures A and B were stored separately for 24 hours at room temperature, and then 2.5 g of Mixture A was mixed with 2.5 g of Mixture B and allowed to cure for 1 hour.

Results (Compressive strengths, flexural strengths and linear shrinkage) are represented in Table 10.

The variants are based on the same formulation as Example 22 and its properties are given also in Table 10.

TABLE 10
Mechanical Properties of unfilled nano-silica containing
resin as a function of PVOH nano-fibers concentration.

| Example 22 with | | | |
|---|---|---|---|
| PVOH Nano-fibers | | | |
| (diameter 250 nm) | | | |
| Addition, % wt. | CS, MPa | FS, MPa | LS, % |
| 0.00 | 100.5 | 195.0 | 2.9 |
| 0.05 | 127.6 | 210.4 | 1.7 |
| 0.10 | 115.4 | 166.9 | 1.6 |
| 0.30 | 111.2 | 148.4 | 2.1 |
| 1.00 | 67.5 | 111.1 | 2.6 |

It will be appreciated by persons skilled in the art that the present invention is not limited by what has been particularly shown and described hereinabove. Rather the scope of the present invention includes both combinations and subcombinations of the features described hereinabove as well as modifications and variations thereof which would occur to a person of skill in the art upon reading the foregoing description and which are not in the prior art.

We claim:

1. A dental material comprising:
a plurality of polymerizable monomers or polymerizable oligomers;
a polymerization initiator, and a
a hyperbranched additive, and
at least one of electrospun nanofiber, electrospun nanosphere or caged macromolecule.

2. The dental material of claim 1 wherein the hyperbranched additive is comprised of hyperbranched molecules or dendritic molecules.

3. The dental mater of claim 2 wherein the dendritic molecule has from 1 to 20 generations of at least one monomeric or polymeric branching chain extender.

4. The dental material of claim 2 where the dendritic molecule is a dendrimer.

5. The dental material of claim 1 wherein the hyperbranched additive is a hyperbranched macromolecule or a dendritic macromolecule having at least one reactive group, said macromolecule built up of hydroxyl units.

6. The dental material of 5 wherein the hydroxyl units are combined with amide molecules with nitrogen atoms as branching points.

7. The dental of claim 1 wherein hyperbranched additive is a hyperbranched macromolecule or a hyperbranched dendritic macromolecule with a reactive group, said reactive group being comprised of hydroxyl, amine, carboylic, ester, amide, sulfide, carboxylate or fatty acid.

8. The dental material of claim 1 wherein the hyperbranched material is hyperbranched polyesteramide or hyperbranched polyester.

9. The dental material of claim 1 wherein the dental material is a composite filling, restorative, adhesive, cement, liner or primer.

10. The dental material of claim 1 wherein at least one of the polymerizable monomers is monofunctional acrylates, multifunctional acrylates, monofunctional methacrylates, or multifunctional methacrylates.

11. The dental material of claim 1 wherein at least one of the polymerizable monomers is methyl methacrylate, bisphenylglycidyl methacrylate (Bis-GMA) triethylene glycol dimethacrylate (TEGDMA), 2-hydroxyethyl methacrylate, hexanediol methacrylate, or dodecanediol dimethacrylate, bisphenol-A-dimethacrylate or 2-hydroxyethylmethacrylate (HEMA).

12. The dental material of claim 1 wherein at least one of the polymerizable monomers contains at least one functional group selected from urethane, amine, acrylic, carboxylic, amide or hydroxyl.

13. The dental material of claim 1 wherein at least one of the polymerizable oligomers is aromatic urethane acrylate, aliphatic urethane acrylate, epoxy-acrylate, urethano-acrylate, or urethane dim-methacrylate.

14. The dental material of claim 1 wherein the electron spun nano-fiber or the electron spun nano-sphere is produced from at least one of silk, cellulose, starch, polyamids. carbon silica, alumina, zirconia, polyurethanes, polyesters, polylactides (PLLA), polyolefins, collagen, polyvinyl alchohol (PVOH), polylacticacid, or polyglycolic.

15. The dental material of claim 1 wherein the caged macromolecule is caged silica or polyhedral oligomeric silsequioxanes (POSS).

16. The dental material of claim 1 further comprised of a filler.

17. The dental material of claim 16 wherein the filler is quartz or silica glass, the silica glass comprised of strontium, barium, zinc, boron, yttrium, aluminoborosilicate glass, strontium-alumino-fluoro-silicate glass or colloidal silica.

18. The dental material of claim 1 further comprised of a cross-linker.

19. The dental material of claim 18 wherein the cross-linker is a multifunctional acrylate.

20. The dental material of claim 1 wherein the polymerization initiator is a chemical initiator or a photo-initiator.

21. The dental material of claim 1 wherein the polymerization initiator comprises camphor quinon, ethyl-4-dimethylaminobezoate (EDB), tertiary amine, benzoyl peroxide, lauryl peroxide, benzophene alpha-diketones, 2,6ditert-butyl-4-methylphenol (BHT), N,N dimethyl-sym-xylidine, diethyl-amine methacrylate or N,N-dimethyl-p-toluidine.

22. The dental material of claim 1 further including a nano-clay.

23. The dental material of claim 21 wherein the nano-clay is alkyl quaternary ammonium bentonite.

24. The dental material of claim 1 wherein further comprising a catalyst, an accelerator for polymerization process, an inhibitor, a stabilizer or a pigment.

25. The dental material of claim 24 wherein the accelerator is N,N-dihydroxyethyl-p-toluidine (DHEPT).
